(19) United States
(12) Patent Application Publication
(10) Pub. No.: US 2006/0051826 A1
(43) Pub. Date: Mar. 9, 2006

(54) Method and device for the incorporating a compound in the pores of a porous material and uses thereof

(75) Inventors: Thu-Hoa Tran-Thi, Allee des Peupliers (FR); Thanh-Toan Truong, Rue du Potager (FR)

Correspondence Address: FOLEY & LARDNER LLP, 1530 PAGE MILL ROAD, PALO ALTO, CA, 94304, US

(73) Assignee: Commissariat A L' Energie Atomique

(21) Appl. No.: 10/517,719

(22) PCT Filed: Jun. 11, 2003
(86) PCT No.: PCT/FR03/01696

§ 371 Date: Dec. 10, 2004

(30) Foreign Application Priority Data
Jun. 11, 2002 (FR) 0207135

(51) Int. Cl. C12Q 1/66; C12N 11/04

(52) U.S. Cl. 435/8; 435/182

(57) ABSTRACT

The invention relates to a process and to a device for the incorporation of a compound in the pores of a porous material chosen from microporous and mesoporous materials obtained by the sol-gel process and to the uses of this process and of this device. The process comprises the evaporation or the sublimation of the compound in a chamber comprising the porous material. Uses: doping of microporous and mesoporous materials obtained by the sol-gel process and in particular of micelle-templated silica materials, in the manufacture of chemical sensors and multisensors, of molecular sieves, of selective membranes for filtration, of stationary phases for chromatography, or of optical or optoelectronic materials.

### TECHNICAL FIELD

The present invention relates to a process for the incorporation of a compound in the pores of a porous material and more specifically of a porous material chosen from microporous and mesoporous materials obtained by the sol-gel process, which will be denoted more simply, in what follows, by sol-gel microporous and mesoporous materials.

It also relates to a device which makes possible the implementation of this process.

The process according to the invention, which is applicable both to an organic compound and to an inorganic compound, results, depending on the operating conditions used, in the incorporation of this compound either in the form of monomers or in the form of aggregates.

In that which precedes and that which follows, the term “monomer” is understood to denote a separate molecule, whereas the term “aggregate” is understood to mean an assemblage of several molecules bonded to one another via noncovalent bonds.

One of the objects of the invention is the incorporation, in the form of monomers, of an organic compound and more particularly of a probe molecule (that is to say, of a detectable molecule capable of interacting specifically with a molecular entity and of revealing the presence thereof and optionally the concentration thereof within a complex mixture) in the pores of a sol-gel microporous or mesoporous material and in particular of a micelle-templated silica material.

A microporous or mesoporous material thus “doped” by a probe molecule has applications in the manufacture of chemical sensors and multisensors and more especially of sensors and multisensors intended for the detection and quantitative determination of atmospheric pollutants.

It can also have applications in numerous other fields, such as the manufacture of molecular sieves, of selective membranes for filtration, of stationary phases for chromatographic operations, in particular exclusion chromatographic operations, or of optical materials, such as optical filters, or optoelectronics, by making use of the nonlinear properties of a doped material.

### STATE OF THE PRIOR ART

Schematically, the sol-gel process consists in forming a colloidal suspension of particles of oxides (or “sol”) by hydrolysis and condensation of a precursor or of a mixture of precursors which are ionic in nature (salts) and/or molecular in nature (alkoxides), in drying the sol, so as to obtain a semi-rigid “gel” by additional condensation of the said precursor or precursors, and in subjecting this gel to a drying and densifying heat treatment.

This process makes it possible to produce highly diverse materials existing in the form of bulky items, of powders, of fibres or of films and in particular of thin microporous and mesoporous films capable of acting, after incorporation of probe molecules, as sensitive layers in chemical sensors and multisensors.

It is generally accepted, in the field of sol-gel materials, that a microporous film comprises pores with a diameter of less than 20 Å (angstroms), whereas a mesoporous material comprises pores with a diameter ranging from 20 Å to 5 μm (microns).

Micelle-templated silica (MTS) materials appeared about ten years ago following the studies by Beck et al. (J. Am. Chem. Soc., 1992, 114, 10834) [1]. These materials are obtained by polycondensating, according to the sol-gel process, networks of metal oxides (in particular silicon alkoxides) in the presence of a surface-active agent, the molecules of which form micelles organized at the nanoscopic scale.

MTS have the distinctive feature of exhibiting a two-fold porosity: this is because the polycondensation of the networks of metal oxides around the micelles of surface-active agent results in the formation of a porous inorganic material, the pores of which form a first unorganized porosity and which comprises a compact and ordered arrangement of organic micelles; by calcination of these micelles, a second porosity, which is organized, contrary to the preceding porosity, and the structure of which depends directly on the size of the micelles and on their three-dimensional arrangement, appears.

Another distinctive feature of MTSs is that they exhibit pores with an adjustable diameter. This is because it is possible to vary the diameter of the pores of the first porosity from approximately 5 to 18 Å, in particular by the choice of the metal oxides used as precursors during the preparation of the MTSs, and to vary the diameter of the pores of the second porosity from approximately 10 to 100 Å, by varying the length of the chain of the surface-active agent or by using an agent capable of swelling the micelles of surface-active agent.

The characteristics of the MTSs which have just been mentioned make it a particularly advantageous material for the preparation of chemical sensors and multisensors and in particular of sensors and multisensors intended to detect and quantify atmospheric pollutants.

This is because: first, the unorganized porosity of the MTSs can act as molecular sieve and can promote the diffusion of the small pollutants or of the gases whose interferences it is desired to study; secondly, the diameter of the pores of the organized porosity can be adjusted so that, after incorporation in these pores of a probe molecule capable of interacting with a family of pollutants, the remaining space corresponds to the kinetic diameter of a specific pollutant of this family. Thus, to the specificity of the probe molecules for a family of pollutants is added a specificity of the diameter of the pores for a specific pollutant of this family. This two-fold specificity makes it possible to avoid, or at the very least to limit, risks of interference between pollutants of the same nature but of different size.

The incorporation of probe molecules in the pores of MTSs for the purpose of the use of the latter as sensitive layers of chemical sensors and multisensors has to satisfy three main requirements:
the probe molecules must not decompose during their incorporation if they are not to lose their reactivity with regard to the pollutants and their ability to act as developers;
they must not be reencountered in the pores in the form of aggregates but only in the form of monomers, the presence of aggregates in the pores simultaneously affecting the diffusion of the pollutants towards the reactive sites, the reactivity of the probe molecules with regard to the pollutants and the properties which render them detectable; and
neither must they be reencountered in the pores in a solvated form, for identical reasons of interfering with diffusion and reaction.

These constraints also exist in the case of the incorporation of a probe molecule in the pores of a porous sol-gel material other than an MTS.

A number of techniques have been provided to date for incorporating a compound in the pores of a microporous or mesoporous material prepared by a process other than the sol-gel process, such as, for example, liquid phase sorption, gas phase sorption, sorption by reaction in the solid state, or ion exchange (see, in this respect, the development published by Schulz-Ekloff et al. in Microporous and Mesoporous Materials, 2002, 51, 91-138 [4]), but processes targeted at incorporating a compound in the pores of a microporous or mesoporous material obtained by the sol-gel process are very limited in number.

In fact, they are essentially processes which consist in adding the compound to the sol before the latter is condensed to give a gel. These processes, which are disclosed in particular in Patents U.S. Pat. No. 5,650,311 [2] and U.S. Pat. No. 5,824,526 [3], have many disadvantages.

This is because, since the sol comprises, as solvents, water and alcohol, they are firstly unsuitable for the incorporation of water-sensitive compounds due to a major risk of hydrolysis of these compounds. They are also unsuitable for the incorporation of hydrophobic compounds which, because of their low solubility in water and alcohol, can only be incorporated in very small amounts and will have a tendency to form aggregates in the sol and, consequently, to be reencountered in this same form in the final material. Furthermore, during the gelling of the sol, the interstitial solvents gradually evaporate, entraining with them molecules of the incorporated compound, thus creating a concentration gradient of this compound in the final material.

The processes disclosed in the abovementioned U.S. patents also present another difficulty, which is that of managing to completely extract the solvents present in the final material, in particular when these solvents interact with the surface of the pores, in order to prevent the incorporated compound from being present in the pores in a solvated form.

Finally, they do not make it possible to monitor the incorporation of the compound while the incorporation is being carried out and it is only after drying the gel and evaporating the residual solvents, that is to say when the compound incorporated has ceased to migrate to the surface of the material, that it becomes possible to confirm whether the incorporation has taken place in a satisfactory manner.

The problem is consequently posed of supplying a process for the incorporation of a compound in the pores of a microporous or mesoporous sol-gel material which:
makes it possible, according to the destination of this material, to incorporate the compound solely in the form of monomers or, in contrast, in the form of aggregates,
employs no solvent or employs solvent only in a very limited way,
does not present a risk of bringing about decomposition of the compound, whether by hydrolysis, thermal decomposition or other route,
can be applied to the greatest possible number of compounds, whether inorganic or organic, hydrophobic or hydrophilic, and the like,
offers the possibility, if desired, of monitoring the incorporation of the compound as the incorporation is carried out, and which, furthermore,
is simple to implement and at an acceptable cost, this being the case both on an industrial scale and on an experimental scale.

This problem is solved by the present invention, which provides a process for the incorporation of a compound in the pores of a porous material chosen from microporous and mesoporous sol-gel materials, as well as a device specifically designed for implementing this process.

### ACCOUNT OF THE INVENTION

The process for the incorporation of a compound in the pores of a porous material according to the invention is characterized in that it comprises the evaporation or the sublimation of this compound in a chamber comprising the said material.

Thus, the process according to the invention is based on the use of a change in physical state of the compound in order to obtain the incorporation thereof in the pores of a porous material, this change consisting either of a passage from the liquid state to the gas state or of a direct passage from the solid state to the gas state.

The temperature at which a compound evaporates or sublimes depends on its pressure, so that it is possible to vary the pressure in order to modify this temperature. This is perfectly illustrated by the P/T phase diagram of a pure body. The evaporation and sublimation temperatures available in the literature correspond, in the absence of contrary indications, to those established at atmospheric pressure and are capable of being substantially lowered by the use of lower pressures, that is to say by the use of vacuum.

According to the invention, the temperature and pressure conditions under which the compound is evaporated or sublimed are chosen, firstly, according to the thermal decomposition temperature of this compound.

This is because it is desirable for the temperature at which the compound is evaporated or sublimed to be lower by at least 30° C. and preferably by at least 50° C. than the temperature at which it decomposes, in order to rule out any risk of thermal decomposition of the said compound during its incorporation in the pores of the porous material.

The thermal decomposition temperatures of a large number of compounds are known, in which case they are generally shown in reference works, such as the Merck Index, twelfth edition, or catalogues of suppliers of chemicals, such as the Aldrich catalogue.

When the decomposition temperature of a compound is not known, then it can be determined, for example by bringing the compound to increasingly high temperatures and by monitoring the temperature at which it is consumed or that at which it loses its properties, for example of absorbance, of fluorescence, of luminescence or other type.

Consequently, whatever the compound which has to be incorporated, it is possible to define, for this compound, a maximum operating temperature, this temperature being, in accordance with the invention, lower by at least 30° C. and preferably by at least 50° C. than the thermal decomposition temperature of the said compound, depending on the margin of safety which it is desired to insert.

The temperature and pressure conditions at which the compound is evaporated or sublimed are chosen, secondly, according to the need and/or the possibility which there is to operate at a temperature lower than that at which it evaporates or sublimes at atmospheric pressure, taking into account in particular the equipment available.

Thus, for example, in the case where the compound exhibits, at atmospheric pressure, a very high evaporation or sublimation temperature (of the order of several hundred degrees) and where it is not possible or not desirable to operate at this temperature, for reasons of equipment, of safety or more simply of operational convenience, then the operation will be carried out under vacuum at a pressure which makes it possible to lower the evaporation or sublimation temperature of the compound to an acceptable value.

On the other hand, in the case where the compound exhibits a high vapour pressure and is capable of evaporating or subliming at a low or moderately high temperature at atmospheric pressure, it will be possible to operate just as easily at this temperature and this pressure as at a lower temperature under vacuum.

Another criterion capable of being taken into consideration for the choice of the temperature and pressure conditions at which the compound is evaporated or sublimed is the rate at which it is desired to incorporate the latter in the pores of the porous material, this rate being itself chosen according to the molecular form (monomers or aggregates) under which it is desired that the compound be present in the pores.

This is because, in order to incorporate the compound in the pores of the porous material solely in the form of monomers, it is desirable to use temperature and pressure conditions capable of allowing the evaporation or the sublimation to be carried out very slowly, so that the diffusion of the compound within the pores is as homogeneous as possible over the whole of the material.

In contrast, when there is no constraint on incorporating the compound in the pores of the porous material in the form of monomers and when the aim of the evaporation or of the sublimation is to incorporate the greatest possible amount of compound in the pores, for example if this material is intended to act as molecular sieves, then it is desirable to use pressure and temperature conditions capable of allowing the pores to be filled as rapidly as possible.

In point of fact, for a given pressure, the higher the temperature of the medium comprising a compound, the faster its rate of evaporation or sublimation, whereas, for a given temperature, the lower the pressure prevailing in the medium comprising a compound, the faster the rate of evaporation or sublimation of this compound.

Consequently, it is possible to adjust the rate at which a compound evaporates or sublimes by varying the temperature or the pressure at which this evaporation or sublimation is carried out.

According to the invention, the aim is to operate, preferably, at a temperature as close as possible to ambient temperature and, in any case, at a temperature which does not exceed 200° C.

For this reason, the compound is evaporated or sublimed, preferably, under vacuum, in which case the process according to the invention comprises:
a) placing the chamber comprising the compound and the porous material under vacuum until the desired vacuum is obtained and, optionally,
b) heating the chamber to the chosen temperature, in order to evaporate or sublime the compound.

Be that as it may, it is the temperature chosen for carrying out the operation which determines whether the compound is evaporated or sublimed. This is because, if it is in a liquid form at this temperature, then it is evaporated, whereas, if it is in a solid form, for example a pulverulent form, or a pasty form, then it is sublimed.

When the compound exists in the form of a paste, the invention provides for its sublimation under vacuum after having separated the agglomerates therefrom by at least partial dissolution of the paste in a volatile solvent which will subsequently be easily removed at ambient temperature during the application of the vacuum in the chamber comprising the compound and the porous material.

According to an advantageous arrangement of the invention, in the case where provision is made to evaporate or sublime the compound under vacuum, the chamber comprising the compound and the porous material is cooled to a temperature of less than or equal to −40° C. immediately before it is placed under vacuum, in order to avoid the latter operation from suddenly sucking up and dispersing the compound throughout the volume of the chamber. This cooling can, for example, be obtained by immersing the chamber in liquid nitrogen or in a bath of dry ice and ethanol.

According to another advantageous arrangement of the invention, in the case where provision is made to evaporate or sublime the compound at a temperature greater than ambient temperature, the chamber comprising the compound and the porous material is heated by immersion in an oil bath maintained at the chosen temperature in order to evaporate or sublime the compound, this being because this heating method guarantees a particularly homogeneous supply of heat.

However, it can also be heated by means of a hot water bath or of electrical heating resistor.

Whatever the method of heating the chamber selected, the porous material is preferably thermally insulated from the wall and from the base of this chamber so that the compound in the gaseous form can condense or solidify once in contact with the walls of the pores of the porous material.

The use of an oil bath is not reserved for the sole case where the compound is intended to be evaporated or sublimed at a temperature greater than ambient temperature. This is because this use can also be envisaged for operating at ambient temperature for the purpose of guaranteeing that this temperature is homogeneous over the perimeter of the chamber and that this chamber is maintained at a constant temperature.

According to the invention, the amount of compound placed under evaporation or sublimation is preferably chosen according to the pore volume of the porous material and the amount of compound which it is desired to incorporate in the pores of this material according to its purpose.

This is because it is possible to determine the optimum amount of compound which has to be present in the pores of the porous material according to the use for which it is reserved.

Thus, for example, in the case where it is intended to act as sensitive layer in a chemical sensor and where, for this reason, the compound to be incorporated is a probe molecule, such as a fluorophore, it is advisable for the amount of fluorophore present in the pores to be sufficiently high for it to be easy to detect it but without being excessively high, in order for the analyte which has to be detected to be able to penetrate into the pores, to interact with the fluorophore, and in order for this interaction to be reflected by a significant variation in the fluorescence emitted by the fluorophore.

The pore volume of a porous material can be measured by low-temperature gas adsorption and desorption techniques. By dividing this pore volume by the volume of a molecule of the compound to be incorporated, the maximum number of molecules of this compound capable of being incorporated per unit of weight of porous material is obtained. Knowing the molecular weight of the compound, the maximum amount by weight of compound capable of being incorporated per unit of weight of porous material and, consequently, of being placed under evaporation or sublimation in order to saturate the pores of 1 g of material can then be easily calculated.

Once this maximum amount is known, it is then possible to determine, by a few experimental trials, the amount of compound to be evaporated or sublimed which is the most suitable for the desired result.

According to another advantageous arrangement of the invention, the process comprises one or more operations for monitoring the incorporation of the compound as the incorporation is carried out. This is because such an arrangement proves to be very useful when it is desired to define the operating conditions (temperature, pressure and duration of the evaporation or the sublimation, amount of compound to be used, and the like) which are the most suitable for producing a specific result (for example, the incorporation of the compound solely in the form of monomers or the achievement of a specific degree of filling of the pores). It also makes it possible to confirm that the incorporation of the compound is correctly carried out with respect to the result desired and, if necessary, to consequently modify the operating conditions.

Preferably, this monitoring is carried out by optical measurements, for example of absorbance, of fluorescence, of luminescence or the like.

According to the invention, the porous material is preferably provided in the form of a block, for example a parallelepipedal block, or of one or more thin layers covering one and/or other of the faces of an inert substrate, such as a quartz or glass slide.

In an alternative form, it is possible, however, to employ the process of the invention with a pulverulent porous material.

The process according to the invention has proved to be just as suitable for the incorporation of compounds in the pores of inorganic materials as in those of organic/inorganic materials or hybrids and in particular in the pores of MTSs, it being possible for all these materials to be based on silicon, vanadium, titanium, tin, zirconium, gallium or a mixture of these.

The process according to the invention exhibits numerous advantages. This is because:
as the evaporation or the sublimation of the compound to be incorporated does not use any solvent, the process prevents both this compound from being reencountered in the pores in a solvated form and a concentration gradient of the compound from being established within the porous material;
in contrast, it results in a very homogeneous distribution of the compound in the pores of the porous material;
it offers the possibility of incorporating the compound in a form (monomers or aggregates) and in an amount which are perfectly suited to the use for which the porous material is intended;
it makes it possible to confirm, as the incorporation is carried out, that the incorporation indeed corresponds to the objectives which have been set and to consequently modify, if appropriate, the operating conditions;
it uses very small amounts of compound, including in the case where it is desired to saturate the pores of the porous material, so that, when the evaporation or the sublimation is carried out in the presence of a heat source, a temperature gradient is not observed in the chamber in which this evaporation or sublimation is carried out;
it is applicable to a very large number of compounds, since, in principle, all compounds can be evaporated or sublimed, and to very different porous materials;
it is simple to employ and offers in particular the possibility of operating at reasonable temperatures; it does not require complex and expensive equipment.

In this respect, another subject-matter of the present invention is a device which makes it possible to employ the process according to the invention and which comprises:
a chamber equipped with an opening,
means for immobilizing at least one sample of porous material in the chamber,
means for thermally insulating this sample from the wall and from the base of the chamber,
means for hermetically sealing the chamber, and
means for connecting the chamber to a vacuum-producing system.

According to a first preferred embodiment of the device, the means for immobilizing the sample of porous material also act as means for thermally insulating it from the wall and from the base of the chamber.

Preferably, these means comprise a support, for example of cylindrical, cubic or frustoconical shape, which is composed of an insulating material, such as Teflon®, which is integrally attached to the base of the chamber and which is equipped with means for holding the said sample in place.

These means for holding in place are composed, for example, of a groove which runs across the face of the support opposite that in contact with the base of the chamber and into which either one of the ends of the sample, if the latter is provided in the form of a block or of one or more thin layers covering one and/or other of the faces of a substrate, can be inserted or the base of a dish comprising the sample, if the latter is provided in a pulverulent form, can be inserted.

The sample or the dish can be held more firmly in position by the presence, along this groove, of one or more elastic or flexible components.

Furthermore, in this first preferred embodiment of the device, the means for hermetically sealing the chamber also act as means for connecting it to the vacuum-producing system, such as a vacuum line.

Advantageously, these means comprise a shutting device composed of a first pipe which is equipped, at one of its ends, with means for hermetically attaching it to the chamber and, at the other of its ends, with a vacuum tap and which carries, in a lateral position, a second pipe terminated by means for connecting it to the vacuum-producing system, the region for butt joining the second pipe on the first being such that the connection between these two pipes can be opened or closed by rotation of the vacuum tap.

Preferably, the chamber is composed of a transparent material, such as quartz or glass, to make possible monitoring, by optical measurements, of the incorporation of the compound as the incorporation is carried out.

Advantageously, the chamber is an optical cell with four faces.

According to another preferred embodiment of the device, the latter additionally comprises means for connecting it, in conjunction with at least one other device as defined above, to a vacuum-producing system.

According to yet another preferred embodiment of the device, the chamber comprises a plurality of pipes each capable of comprising at least one sample of porous material, each pipe being equipped with means for immobilizing the sample which it comprises and with means for thermally insulating it from the other pipes, from the base of the chamber and, if appropriate, from the wall of this chamber.

In this last preferred embodiment of the device, the means for immobilizing the sample of porous material also act to thermally insulate it from the base of the chamber.

Preferably, these means comprise, here also, a support composed of an insulating material which is integrally attached to the base of the chamber and which is equipped with means for holding the said sample in place.

Furthermore, the means for thermally insulating the sample of porous material from the other pipes and, if appropriate, from the wall of the chamber are composed of the wall of the pipe in which it is found, this wall being formed of an insulating material, such as Teflon®.

In this last preferred embodiment of the device, the means for hermetically sealing the chamber also act as means for connecting it to the vacuum-producing system and comprise, on the one hand, a lid capable of being hermetically attached to the chamber and, on the other hand, a shutting device composed of a first pipe which is equipped, at one of its ends, with means for hermetically attaching it to the lid and, at the other of its ends, with a vacuum tap and which carries, in a lateral position, a second pipe terminated by means for connecting it to the vacuum-producing system, the region for butt joining the second pipe on the first being such that the connection between these two pipes can be opened or closed by rotation of the vacuum tap.

An additional subject-matter of the invention is the use of a process or of a device as defined above for incorporating an organic compound in the form of monomers in the pores of a porous material chosen from microporous and mesoporous sol-gel materials and more particularly in the pores of a micelle-templated silica (MTS) material, it being possible for all these materials to be based on silicon, vanadium, titanium, tin, zirconium, gallium or also a mixture of these.

Mention may be made, as examples of MTSs, of those under the names M41S, MCM-41, MCM-48, SBA, HMS, MSU, FSM-16, PCH and ZSM.

Preferably, the porous material is provided in the form of a block or of one or more thin layers covering one and/or other of the faces of an inert substrate, while the compound is a probe molecule capable of detecting and optionally quantifying an analyte, that is to say, in practice, a label or a ligand coupled to a label.

In the context of the invention, the term “label” is understood to mean a molecule possessing a particular physical property which renders it detectable and identifiable. Furthermore, the term “ligand” is understood to mean a molecule capable of interacting with an analyte by collision or by forming a complex with it via a physical or chemical bond.

Thus, depending on the ability of the label to interact with the analyte, it can be used alone or in combination with a ligand capable itself of interacting with the said analyte.

Mention may be made, as examples of labels capable of being used in the context of the invention, of fluorophores, such as Bodipy, 1,3-diphenyl-1,3-dipropanedione, dibenzoylmethanatoboron difluoride and its derivatives (naphthoyl-benzoyl, biphenylcarbonyl-benzoyl, biphenylcarbonyl-naphthoyl, methoxybenzoyl-benzoyl), phenylhydrazine and its nitrated and chlorinated derivatives, O-pentafluorobenzylhydroxylamine, anthracene and its derivatives, bianthryl and its derivatives, pyrene and its derivatives, pyrenol, pyranine, fluorescein, Oregon green, rhodamine and its derivatives, cyanine and its derivatives, porphyrins, phthalocyanines, porphyrazines, or tetracyanoquinodimethane and its derivatives; luminophores, such as luminol and luciferin; or chromophores, such as xanthene, anthraquinone, monoazo compounds, diazo compounds and triphenylmethane.

A microporous or mesoporous material obtained by the sol-gel process, the pores of which include a probe molecule in the form of monomers, is of great advantage in the manufacture of chemical sensors and, a fortiori, of chemical multisensors (a multisensor being composed of a plurality of sensors) intended to detect or to quantitatively determine a combination of analytes and more especially of atmospheric pollutants (CO, CO₂, NO₂, NO, SO₂, CH₂O and other aldehydes, benzene, toluene, xylenes, ethylbenzene, and the like) or of gas molecules used in the field of microelectronics (Cl₂, BCl₃, AlCl₃, and the like).

Thus, for example, a material including phenylhydrazine, one of its nitrated or chlorinated derivatives or O-pentafluorobenzylhydroxylamine as probe molecule can be used to collect formaldehyde or other aldehydes present in the gaseous form in the air.

A material including 1,3-diphenyl-1,3-dipropanedione as probe molecule can be used to collect the gases BCl₃ and AlCl₃.

A material including dibenzoylmethanatoboron difluoride or one of its derivatives (naphthoyl-benzoyl, biphenylcarbonyl-benzoyl, biphenylcarbonyl-naphthoyl, methoxybenzoyl-benzoyl) as probe molecule can be used to collect benzene and its substituted derivatives, while a material including a metallated phthalocyanine or porphyrin as probe molecule can be used to collect CO, NO and/or NO₂.

A further subject-matter of the invention is thus the use of a process or of a device as defined above in the manufacture of a chemical sensor or multisensor, in particular for the detection or the quantitative determination of atmospheric pollutants.

In addition to the preceding arrangements, the invention also comprises other arrangements which will emerge from the remainder of the description which follows, which is given by way of illustration and without implied limitation, with reference to the appended drawings.

### BRIEF DESCRIPTION OF THE DRAWINGS

FIG. 1 is a diagrammatic view in vertical cross section of a device according to the invention in a first embodiment.

FIG. 2 is a diagrammatic view in vertical cross section of a device according to the invention in a second embodiment.

FIG. 3 is a partial diagrammatic view in vertical cross section of a device according to the invention in a third embodiment.

FIG. 4 is a graph illustrating the variation in the absorbance (solid line) and that in the area of fluorescence (dashed line) of dibenzoylmethanatoboron difluoride (DBMBF₂), after incorporation in the pores of an MTS by the process according to the invention, as a function of the duration of the sublimation of this compound when this sublimation is carried out at a pressure of 5.33×10⁻³ Pa and a temperature of 25° C.

FIG. 5 is a graph illustrating the variation in the area of fluorescence of DBMBF₂, after incorporation in the pores of an MTS by the process according to the invention, as a function of the absorbance of this compound when its sublimation is carried out at a pressure of 5.33×10⁻³ Pa and a temperature of 25° C.

FIG. 6 is a graph illustrating the change in the fluorescence spectrum of DBMBF₂, after incorporation in the pores of an MTS by the process according to the invention, as a function of the duration of the sublimation of this compound when this sublimation is carried out at a pressure of 5.33×10⁻³ Pa and a temperature of 25° C.

FIG. 7 is a graph illustrating the change in the absorption spectrum of DBMBF₂, after incorporation in the pores of an MTS by the process according to the invention, as a function of the duration of the sublimation of this compound when this sublimation is carried out at a pressure of 5.33×10⁻³ Pa and a temperature of 75° C.

FIG. 8 is a graph illustrating the variation in the absorbance of DBMBF₂, after incorporation in the pores of an MTS by the process according to the invention, as a function of the duration of the sublimation of this compound when this sublimation is carried out at a pressure of 5.33×10⁻³ Pa and a temperature of 75° C.

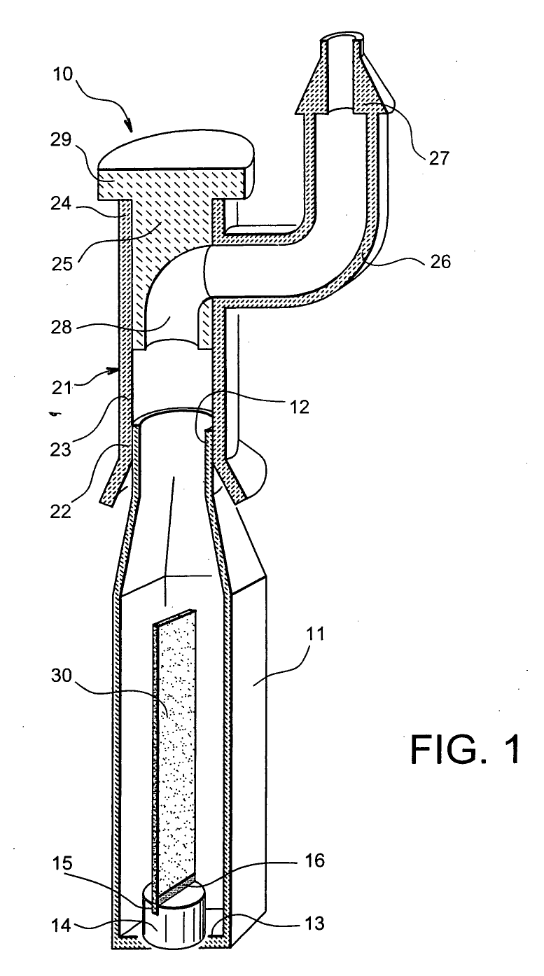

In FIGS. 1 to 3, the same references are used to denote the same components.

### DETAILED ACCOUNT OF EMBODIMENTS OF A DEVICE ACCORDING TO THE INVENTION

Reference is made first of all to FIG. 1, which shows a device 10 according to the invention in an embodiment specifically designed to incorporate a compound in the pores of a porous material by the process according to the invention while monitoring this incorporation by optical measurements. Furthermore, this embodiment is designed to just as easily carry out an evaporation as a sublimation, this being the case whatever the temperature chosen and the pressure chosen.

As seen in FIG. 1, this device comprises two components, namely a cell 11 and a removable shutting device 21 which is designed to be able to be hermetically attached to the cell 11 by fitting a circular male ground joint 12, exhibited by the latter, into a circular female ground joint 22 which the shutting device 21 comprises.

The cell 11, in which the porous material is intended to be placed, has a square cross section and is made of a transparent material, preferably quartz, in order for the optical measurements to be able to be carried out as evaporation or sublimation of the compound takes place.

On the opposite side from the male ground joint 12, the cell 11 exhibits a flat base 13, at the centre of which is attached a solid cylinder 14, the face of which opposite that situated in contact with this base has running across its diameter a groove 15 equipped with a flexible strip 16, for example made of metal.

As seen in FIG. 1, the groove 15 is intended to house the end of one or of two samples 30 of the porous material which has to be treated and to ensure, in conjunction with the flexible strip 16, that they are held immobile in the cell 11.

A sample of porous material capable of being treated using the device 10 can be provided either in the form of a block, or in the form of a thin layer covering one of the faces of a substrate of the quartz slide, glass slide or analogous type, in which case two identical or different samples can be joined together by mutual contact of their faces opposite those covered by the porous material and can be held in place as such by virtue of the groove 15 and the flexible strip 16, or alternatively in the form of two thin layers each covering one of the faces of the same substrate.

In accordance with the invention, the cylinder 14 is composed of a material capable of thermally insulating the sample or samples 30 of porous material when the cell 11 is cooled or heated. This insulating material is, for example, Teflon®.

The shutting device 21 has a two-fold function: this is because it is used, on the one hand, to hermetically seal the cell 11 when this is desired and, on the other hand, to connect this cell to a vacuum line (not represented in FIG. 1), if it is desired to place it under vacuum. For this reason, it is provided in the form of a straight pipe 23, one end of which corresponds to the circular female ground joint 22 while the other end 24 is equipped with a vacuum tap 25. It carries, in a lateral position, an angled pipe 26 which itself ends in a conical male ground joint 27 capable of being fitted into a conical female ground joint of the vacuum line and whose region for butt joining on the straight pipe 23 is situated facing the region for butt joining the internal shaft 28 of the vacuum tap 25 on this same pipe when this tap is in the open position. Thus, the connection between the pipes 23 and 26 can be alternatively opened or closed by rotation of the key 29 of the vacuum tap 25.

The device 10 is extremely simple to use. After having deposited at the base of the cell 11, around the bottom of the cylinder 14, the compound which has to be evaporated or sublimed, for example using a Pasteur pipette or a flexible Teflon® pipe, one of the ends of the sample or samples of the porous material which has to be treated is inserted into the groove 15 of the cylinder 14.

The shutting device 21 is attached to the cell 11, the vacuum tap 25 being in the closed position.

Either the evaporation or the sublimation of the compound is expected to be carried out without use of the vacuum, in which case this evaporation or sublimation is carried out directly by heating the cell to the chosen temperature in order to evaporate or sublime the compound.

Or the evaporation or the sublimation of the compound is expected to be carried out under vacuum, in which case the device 10 is connected to the vacuum line and the cell 11 is immersed in liquid nitrogen or in a mixture of dry ice and alcohol for the time needed to bring its internal temperature to a value of less than −40° C. and thus to prevent, when being placed under vacuum, the compound from being suddenly sucked up and dispersed throughout the volume of the cell 11. The vacuum tap 25 is then opened and the vacuum is allowed to become established in the cell 11. Once the desired vacuum is obtained, this tap is closed and the evaporation or the sublimation of the compound is allowed to take place, optionally in the presence of a heat source, if the temperature chosen for carrying out this evaporation or sublimation is greater than ambient temperature.

It results from the above that the device 10 represented in FIG. 1 does not make it possible to treat more than two samples of a porous material simultaneously.

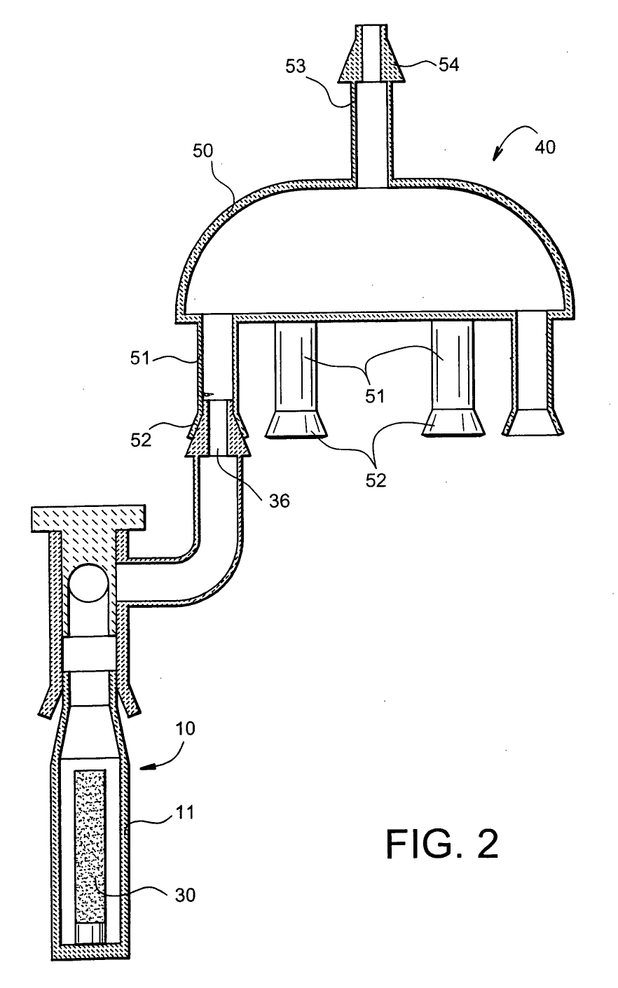

Consequently, FIG. 2 illustrates a device 40 according to the invention in a second embodiment which makes it possible to treat, in parallel, from six to twelve samples of a porous material (depending on whether concerns blocks or thin layers deposited on a substrate) while making it possible to monitor this treatment by optical measurements.

To do this, this device 40 comprises six devices 10 as illustrated in FIG. 1, as well as a “cow's udder” adapter 50 which makes it possible to connect these six devices to one and the same vacuum line (not represented in FIG. 2), for the case where it is desired to establish the vacuum in the cells 11 of the devices 10.

For this reason, the adapter 50 comprises, at its base, six connecting pieces 51 which each end in a conical female ground joint 52 capable of being fitted over the conical male ground joint 36 of the devices 10 and, at its top, a connecting piece 53 equipped at its end with a conical male ground joint 54 capable of being fitted into a conical female ground joint of the vacuum line.

Because each device 10 is equipped with a shutting device which makes it possible to hermetically seal the cell 11 which it comprises, which eliminates any risk of contamination from one device to another, the device 40 offers the possibility of simultaneously treating samples of porous material with different compounds, provided that these treatments can be carried out at the same temperature, it being possible for the pressure to vary from one cell to another.

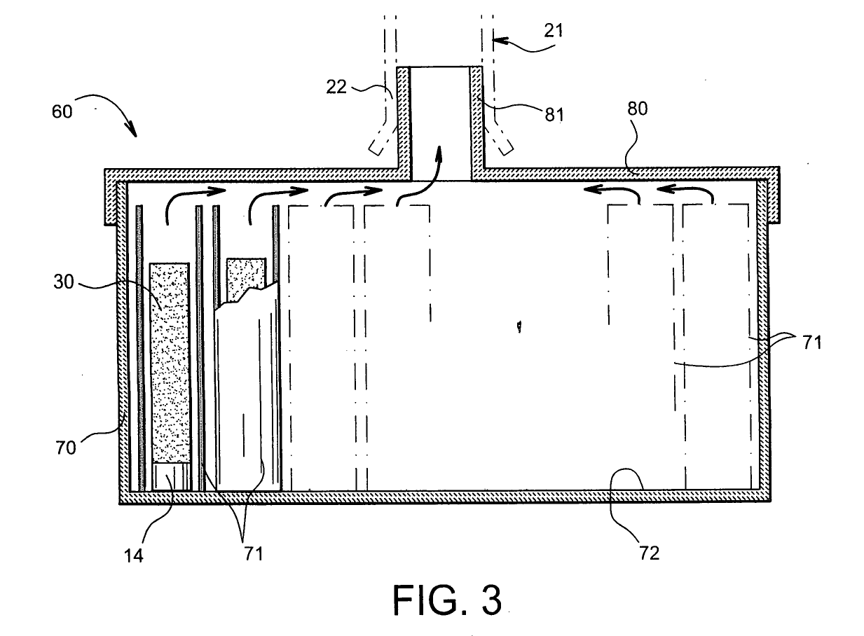

FIG. 3 shows a device 60 according to the invention in yet another embodiment which is specifically designed to make possible the simultaneous treatment of a large number of samples, for example on an industrial scale, but without monitoring this treatment by optical measurements.

As seen in FIG. 3, the device 60 comprises three components, namely a cylindrical container 70, a removable lid 80 which is designed to able to be hermetically attached over the container 70 by fitting and a shutting device 21, also removable, which is intended to be hermetically attached to the lid 80 by interaction of a circular male ground joint 81 which the latter exhibits with a circular female ground joint 22 which the shutting device 21 comprises.

As the latter has the same structure and the same function as the shutting device 21 which can be seen in FIG. 1, namely to provide hermetic sealing of the container 70, on the one hand, and to make possible its connection to a vacuum line, on the other hand, it is only partially represented in FIG. 3.

The container 70 includes a plurality of pipes 71 positioned in rows and having bases formed by the base 72 of this container. As these pipes are intended to house the samples 30 of porous material which have to be treated, their wall 73 is composed of a material capable of thermally insulating them from one another, such as Teflon®.

A solid cylinder 14 having the same structure and the same function as the cylinder 14 represented in FIG. 1 is found at the centre of the base of each pipe. Thus, the samples 30 of porous material are thermally insulated both by the wall of the pipes 71 in which they are housed and by the cylinder 14 present in these pipes. Furthermore, this cylinder makes it possible to channel the gas resulting from the evaporation or the sublimation in the pipes 71 and to promote its diffusion along the samples of porous material.

The device 60 is used according to the same principle as the preceding devices. However, as the pipes 71 do not comprise any means of individual closure, they can only be used to treat the combined samples with one and the same compound or otherwise the pipes will contaminate one another, in contrast to the device 40 of FIG. 2.

### DETAILED ACCOUNT OF EMBODIMENT OF A PROCESS ACCORDING TO THE INVENTION

### EXAMPLE 1

### Incorporation of an Organic Compound in the Pores of an MTS in the Form of Monomers

In this example:
The organic compound is a fluorophore, in the case in point dibenzoylmethanatoboron difluoride (DBMBF₂), which is typically a compound which hydrolyses in the presence of traces of water and which is therefore difficult to incorporate in the pores of a porous material in a solvent medium.
The MTS is a mesoporous silica of the MCM-41 family; its organized porosity is therefore composed of hollow spherical pores organized in hexagonal structures. The diameter of these pores is 25 Å. In the present example, it is used in the form of two thin layers, with a thickness of 300 nm (nanometres), each covering one of the faces of a quartz slide measuring 31 cm in length by 8 cm in width and 1 mm in thickness.
The incorporation of the DBMBF₂ in the pores of the mesoporous silica is carried out by sublimation of the DBMBF₂ at a temperature of 25° C. and under a pressure of 5.33×10⁻³ Pa (4×10⁻⁵ torr) using a device as illustrated in FIG. 1, the cell 11 of which is made of quartz, with a length of 42 cm and a side width of 10 cm, and the cylinder 14 of which, situated inside this cell, is made of Teflon®.

To do this, the quartz slide coated with the two thin layers of mesoporous silica is introduced into the cell 11 of the device 10 and is inserted into the groove of the cylinder 14 situated inside the cell. Approximately 0.5 mg of DBMBF₂ is deposited around the bottom of this cylinder. The shutting device 21 is attached to the cell 11 and the device is connected to a vacuum line, the vacuum tap 25 being in the closed position. The cell 11 is cooled by immersing it in liquid nitrogen for 3 to 4 min.

The vacuum tap 25 is then opened very gently. After slow pumping and the production of a vacuum of 5.33×10⁻³ Pa, this tap is reclosed. The device 10 is disconnected from the vacuum line and the cell 11 is immersed in an oil bath heated to 25° C., the level of the oil being adjusted in order to heat the wall of the cell 11 over its entire height in order to prevent the gaseous DBMBF₂ from condensing on the said wall, thus rendering the optical measurements difficult.

Sublimation is allowed to take place for 11 hours.

The incorporation of the DBMBF₂ in the pores of the mesoporous silica is followed by measuring, regular intervals (every hour), the absorbance at 350 nm and the fluorescence emitted by this compound with regard to these thin layers at, this being carried out using a Perkin® Lambda 900 spectrophotometer and a SPEX® Fluorolog 2 spectrofluorimeter.

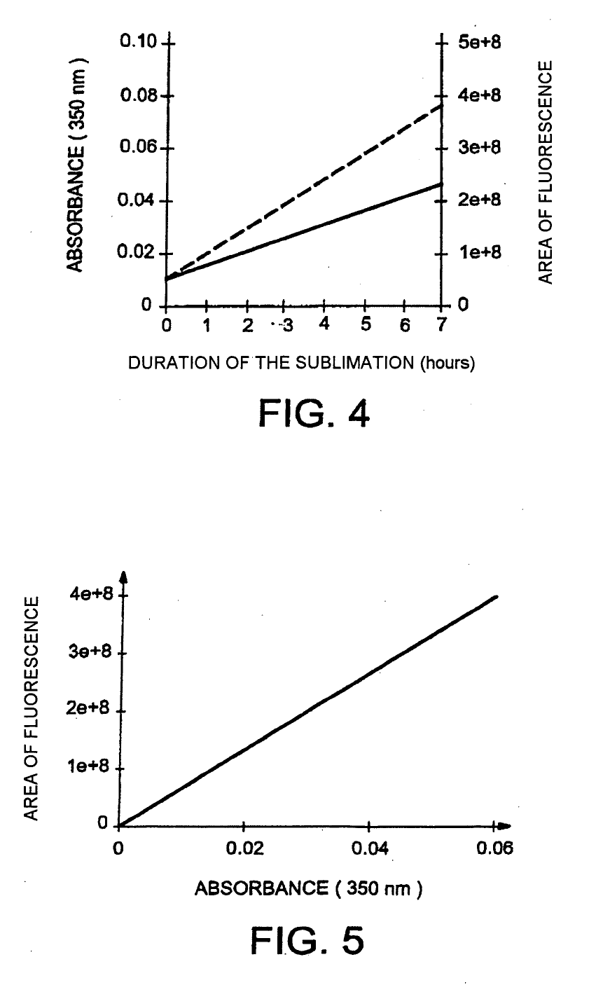

The results of these measurements are illustrated in FIGS. 4 to 6.

FIG. 4 represents the variation in the absorbance at 350 nm (solid line) of the DBMBF₂ and that of the area of fluorescence (dashed line) of the DBMBF₂ obtained for an excitation wavelength of 350 nm, as observed during the first 7 hours of the sublimation.

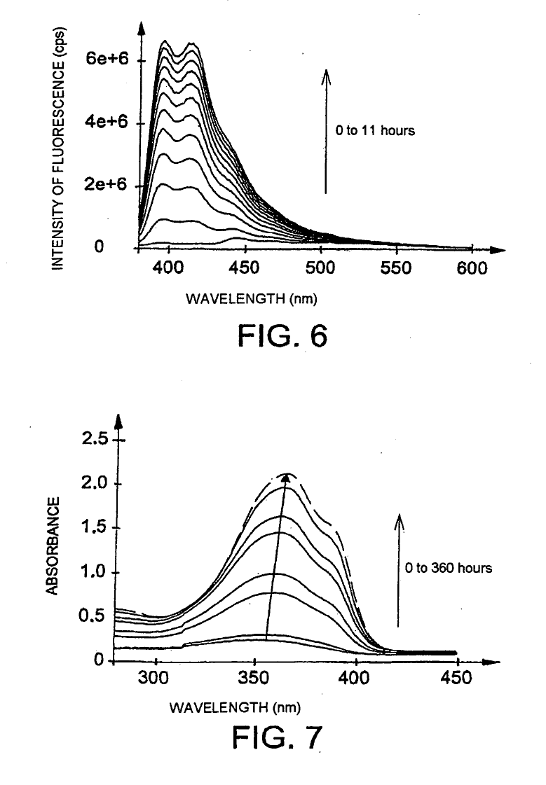

FIG. 5, for its part, represents the variation in the area of fluorescence of the DBMBF₂ as a function of the absorbance at 350 nm of this compound, while FIG. 6 represents the change in the fluorescence spectrum of the DBMBF₂ as observed throughout the duration of the sublimation.

These figures show that:
the absorbance and the area of fluorescence of the DBMBF₂ vary linearly as a function of the duration of the sublimation for a given temperature (in the case in point, 25° C.) (FIG. 4),
the area of fluorescence of the DBMBF₂ also varies linearly as a function of the absorbance of this compound (FIG. 5), and
the fluorescence spectrum of the DBMBF₂ remains unchanged throughout the duration of the sublimation.

These results reflect the presence, in the pores of the mesoporous silica thus treated, of a single fluorescent entity corresponding to DBMBF₂ monomers.

Furthermore, an absorbance at 350 nm equal to 0.065 is obtained after subliming the DBMBF₂ for 7 hours.

By way of comparison, tests carried out by the inventors have shown that, when this compound is incorporated in the pores of the same mesoporous silica by dipping identical samples to those used above in a 10⁻⁵ M solution of DBMBF₂ in cyclohexane devoid of traces of water, it proves to be necessary to dip for 10 days in order to obtain, after drying in the open air, an absorbance at 350 nm of 0.065.

### EXAMPLE 2

### Incorporation of an Organic Compound in the Pores of an MTS in the Form of Aggregates

This example differs from the preceding example only in that the sublimation of the DBMBF₂ is carried out at 70° C., in order to increase the rate of incorporation of this compound, and for 15 days.

In this example, the incorporation of the DBMBF₂ in the thin layers of mesoporous silica is also followed by measuring, at various intervals (1 h 30, 3 h, 18 h 30, 21 h 30, 24 h, 26 h, 42 h, 44 h, 46 h 30, 48 h 30, 50 h 30, 65 h, 67 h, 70 h, 72 h, 74 h, 89 h 45, 91 h 30, 93 h 30, 96 h 30, 98 h 30, 352 h 30, 354 h 30, 356 h 30, 358 h 30, 360 h 30 and 362 h 30), the absorbance of this compound between 300 and 450 nm with regard to these thin layers.

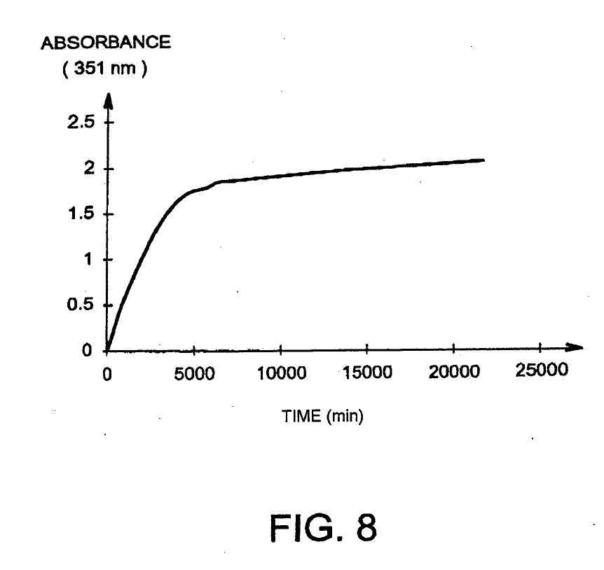

The results of these measurements are illustrated in FIGS. 7 and 8.

FIG. 7, which represents the change in the absorption spectrum of the DBMBF₂ as a function of the duration of the sublimation of this compound, shows that, at the beginning of the sublimation, the appearance of this absorption spectrum remains unchanged but that its intensity increases, reflecting filling of the pores of the mesoporous silica by the DBMBF₂. Subsequently, a slight shifting in the spectrum is observed, which becomes more marked over time and which corresponds to the formation of aggregates.

FIG. 8, which represents the variation in the absorbance at 351 nm of of DBMBF₂ as a function of the duration of the sublimation of this compound, shows that, for a fixed wavelength (351 nm), this variation follows a first linear rule and then curves to reach a plateau value corresponding to saturation of the pores of the mesoporous silica by the DBMBF₂.

The measurement of the absorbance over the entire length of the thin layers of mesoporous silica indicates homogeneous incorporation of the DBMBF₂ in the pores of these thin layers.

### DOCUMENTS CITED

E1] Beck et al., J. Am. Chem. Soc., 1992, 114, 10834
[2] U.S. Pat. No. 5,560,311
[3] U.S. Pat. No. 5,824,526
[4] Schulz-Ekloff et al., Microporous and Mesoporous Materials, 2002, 51, 91-138.

1. Process for the incorporation of a compound in the pores of a porous material chosen from microporous and mesoporous materials obtained by the sol-gel process, characterized in that it comprises the evaporation or the sublimation of this compound in a chamber comprising the said material.

2. Process according to claim 1, characterized in that the temperature at
which the compound is evaporated or sublimed is lower by at least 30° C. and preferably by at least 50° C. than its thermal decomposition temperature.

3. Process according to claim 1, characterized in that the temperature at which the compound is evaporated or sublimed is at most equal to 200° C.

4. Process according to claim 1, characterized in that the compound is evaporated or sublimed under vacuum, in which case it comprises:
a) placing the chamber comprising the compound and the porous material under vacuum until the desired vacuum is obtained and, optionally,
b) heating the chamber to the chosen temperature, in order to evaporate or sublime the compound.

5. Process according to claim 4, characterized in that the chamber comprising the compound and the porous material is cooled to a temperature of less than or equal to −40° C. immediately before it is placed under vacuum.

6. Process according to claim 1, characterized in that, in order to evaporate or sublime the compound at a temperature greater than ambient temperature, the chamber comprising the compound and the porous material is heated by immersion in an oil bath maintained at the chosen temperature in order to evaporate or sublime the compound.

7. Process according to claim 1, characterized in that the porous material is thermally insulated from the wall and from the base of the chamber.

8. Process according to claim 1, characterized in that it comprises one or more operations for monitoring the incorporation of the compound in the pores of the porous material.

9. Process according to claim 8, characterized in that the monitoring is carried out by optical measurements.

10. Process according to claim 1, characterized in that the porous material present in the chamber is in the form of a block or of one or more thin layers covering one and/or other of the faces of an inert substrate.

11. Process according to claim 1, characterized in that the porous material is an inorganic or organic/inorganic hybrid material.

12. Process according to claim 1, characterized in that the porous material is a micelle-templated silica (MTS) material.

13. Device (10, 40, 60) for the incorporation of a compound in the pores of a porous material chosen from microporous and mesoporous materials obtained by the sol-gel process, characterized in that it comprises:
a chamber (11, 70) equipped with an opening,
means for immobilizing at least one sample of porous material in the chamber,
means for thermally insulating this sample from the wall and from the base of the chamber,
means for hermetically sealing the chamber, and
means for connecting the chamber to a vacuum-producing system.

14. Device according to claim 13, characterized in that the means for immobilizing the sample of porous material also act as means for thermally insulating it from the wall and from the base of the chamber.

15. Device according to claim 14, characterized in that the means for immobilizing the sample of porous material and for thermally insulating it from the wall and from the base of the chamber comprise a support (14) composed of an insulating material which is integrally attached to the base (13) of the chamber and which is equipped with means (15, 16) for holding the said sample in place.

16. Device according to claim 13, characterized in that the means for hermetically sealing the chamber also act as means for connecting it to the vacuum-producing system.

17. Device according to claim 16, characterized in that the means for hermetically sealing the chamber and for connecting it to the vacuum-producing system comprise a shutting device (21) composed of a first pipe (23) which is equipped, at one of its ends, with means (22) for hermetically attaching it to the chamber and, at the other of its ends, with a vacuum tap (25) and which carries, in a lateral position, a second pipe (26) terminated by means (27) for connecting it to the vacuum-producing system, the region for butt joining the second pipe on the first being such that the connection between these two pipes can be closed or opened by rotation of the vacuum tap.

18. Device according to claim 13, characterized in that the chamber (11) is composed of a transparent material.

19. Device according to claim 18, characterized in that the chamber (11) is an optical cell with four faces.

20. Device (40) according to claim 13, characterized in that it additionally comprises means (50) for connecting it, in conjunction with at least one device as defined in any one of claims 13 to 19, to a vacuum-producing system.

21. Device (60) according to claim 13, characterized in that the chamber comprises a plurality of pipes (71) each capable of comprising at least one sample of porous material, each pipe being equipped with means for immobilizing the sample which it comprises and with means for thermally insulating it from the other pipes, from the base (72) of the chamber and, if appropriate, from the wall of this chamber.

22. Device according to claim 21, characterized in that the means for immobilizing the sample of porous material also act to thermally insulate it from the base (72) of the chamber.

23. Device according to claim 22, characterized in that the means for immobilizing the sample of porous material and for thermally insulating it from the base (72) of the chamber comprise a support (14) composed of an insulating material which is integrally attached to the base (72) of the chamber and which is equipped with means (15, 16) for holding the said sample in place.

24. Device according to claim 20, characterized in that the means for thermally insulating the sample of porous material from the other pipes (71) and, if appropriate, from the wall of the chamber are composed of the wall (73) of the pipe (71) in which it is found, this wall being formed of an insulating material.

25. Device according to claim 19, characterized in that the means for hermetically sealing the chamber also act as means for connecting it to the vacuum-producing system.

26. Device according to claim 25, characterized in that the means for hermetically sealing the chamber and for connecting it to the vacuum-producing system comprise a lid (80) capable of being hermetically attached to the chamber as well as a shutting device (21) composed of a first pipe (23) which is equipped, at one of its ends, with means (22) for hermetically attaching it to the lid and, at the other of its ends, with a vacuum tap (25) and which carries, in a lateral position, a second pipe (26) terminated by means (27)-for connecting it to the vacuum-producing system, the region for butt joining the second pipe on the first being such that the connection between these two pipes can be opened or closed by rotation of the vacuum tap.

27. Use of a process according to claim 1 for incorporating an organic compound in the form of monomers in the pores of a porous material chosen from microporous and mesoporous materials obtained by the sol-gel process.

28. Use according to claim 27, characterized in that the porous material is a micelle-templated silica (MTS) material.

29. Use according to claim 27, characterized in that the porous material is provided in the form of a block or of one or more thin layers covering one and/or other of the faces of an inert substrate.

30. Use according to any one of claim 27, characterized in that the compound is a label or a ligand coupled to a label.

31. Use according to claim 30, characterized in that the compound is chosen from fluorophores, luminophores and chromophores.

32. Use of a process according claim 1 in the manufacture of a chemical sensor or multisensor.

33. Use according to claim 32, characterized in that the chemical sensor or multisensor is intended for the detection or the quantitative determination of atmospheric pollutants or of gases used in the microelectronics industry.
